(19) United States
(12) Patent Application Publication
(10) Pub. No.: US 2014/0256919 A1
(43) Pub. Date: Sep. 11, 2014

(54) Device for Carrying Out Chemical and/or Biochemical Processes

(71) Applicant: ROBERT BOSCH GMBH, Stuttgart (DE)

(72) Inventors: Arne Kloke, Freiburg (DE); Juergen Steigert, Stuttgart (DE)

(73) Assignee: ROBERT BOSCH GMBH, Stuttgart (DE)

(21) Appl. No.: 14/195,364

(22) Filed: Mar. 3, 2014

(30) Foreign Application Priority Data
Mar. 5, 2013 (DE) 10 2013 203 682.5

(51) Int. Cl. C07K 1/22; B01L 3/00

(52) U.S. Cl. CPC C07K 1/22; B01L 3/5082
USPC 530/413; 422/533

(57) ABSTRACT

A device for carrying out chemical and/or biochemical processes comprises at least two bodies which are arranged axially on top of one another and have in each case at least one cavity. The bodies are rotatable against one another on the basis of a centrifugal force or a similarly acting force. At least one cavity is provided with a membrane, the permeability of which to liquids is dependent on the acting centrifugal force or the similarly acting force.

### CLAIM OF PRIORITY

This application claims priority under 35 U.S.C. §119 to patent application no. DE 10 2013 203 682.5, filed on Mar. 5, 2013 in Germany, the disclosure of which is incorporated herein by reference in its entirety.

### BACKGROUND

The present disclosure relates to a device for carrying out chemical and/or biochemical processes and also to use of a reaction container.

Carrying out biochemical or chemical processes, for example in connection with the purification of particular molecules and/or with the analysis and characterization of particular molecules, is based substantially on the handling of liquids. Traditionally, various aids, in particular pipettes and various reaction vessels, are used for this purpose in order to be able to carry out the various processes in the case of manual handling using various laboratory instruments. For many reactions, automated systems are already available, with liquid-handling robots or other specific instruments being used for example. In addition, so-called Lab-on-a-Chip systems make it possible to carry out many biochemical processes in a fully automated manner. These are microfluidic systems which combine the entire functionality of a macroscopic laboratory on a plastic substrate having approximately only the size of a credit card. In addition to the plastic substrate with various channels, reaction chambers, etc., it is necessary to have stored reagents and various active components, for example valves or pumps, and also further actuation, detection and control units.

For many chemical or biochemical processes, centrifugation is used. By means of the centrifugal forces generated in this operation, it is possible to carry out substance separation on the basis of a difference in density between the various components of a mixture. In addition, the centrifugation allows transport of liquids from a radially more internal process stage to a radially more external process stage.

The German patent application DE 10 2010 003 223 A1 describes a system which comprises a device which is intended for use in a centrifugal rotor. Here, two or more turret-type bodies are arranged axially on top of one another. The turrets contain one or more cavities, more particularly reaction chambers, channels and optionally further structures for carrying out processes. A change in acceleration of the centrifuge activates an integrated mechanism which acts in the manner of a ballpoint-pen mechanism. As a consequence of the centrifugal force, the bodies move radially outward, the bodies being rotated against one another by means of a tooth system and an integrated restoring means. As a result, individual cavities can be interconnected to one another. Furthermore, orientation-dependent opening of individual cavities or vessels of a body is possible, one side of the vessel being provided with, for example, a puncturable film. With the aid of a spike on the other body, the film is pierced through by the movement of the bodies against one another. This makes it possible to achieve controlled conductance of fluid in the device. For example, it is possible to realize guidance of fluid from storage chambers across interconnected processing chambers right up to collection cavities for the processed liquids.

Many biochemical methods used small spherules, known as beads, in order, for example, to carry out purification of particular molecules. Certain interaction partners of a molecule to be purified can be coupled or applied to the beads. During incubation of said beads with a mixture containing the molecule to be purified, the molecule binds to the interaction partner and is thus coupled to the beads. The beads can then be isolated from the rest of the solution by, for example, sedimentation or on the basis of magnetic properties, and so the molecule can be purified very efficiently.

The international patent application WO 01/85341 A1 describes a microfluidic device in the form of a chip having a compartment containing beads which are retained using a filter. Said chip can be used for bead-based purification methods.

### SUMMARY

The device according to the disclosure is intended for carrying out, more particularly carrying out in an automated manner, chemical and/or biochemical processes, for example for purification of proteins and/or of genetic material, more particularly DNA or RNA. To carry out the processes, a certain fluid flow is set in the device, a centrifugal force being exploited for this purpose. The device can be intended for insertion into a rotor of a centrifuge. The device comprises at least two bodies which are arranged axially on top of one another and have in each case at least one cavity. A first body can, for example, be provided with multiple cavities intended for various reagents, solutions or mixtures required for the particular process. The other body can be provided with one or more cavities acting as, for example, reaction spaces. The at least two bodies are rotatable against one another on the basis of a centrifugal force or a similarly acting force, making it possible, in a settable manner, to bring, in each case, a cavity of one body in close proximity to a cavity of the other body, and so fluid flow can take place. Instead of inserting the device into the rotor of a centrifuge and exerting centrifugal force, it is also possible, for example, to apply pressurized air to the device. As a result, rotating of the bodies can likewise be achieved, and the advantages of a stationary system can be utilized, for example various parameters can be adjusted more easily than in a centrifuge. Where centrifugal forces are mentioned hereinafter, it is possible in principle to replace the centrifugal forces with similarly acting forces triggered by, for example, pressurized air. According to the disclosure, at least one cavity of a body is provided with a membrane, the permeability of which to liquids is dependent on the acting centrifugal force or the similarly acting force. By using such a membrane in the device, a valve action is achieved. This means that the fluid flow through the cavity having the membrane is achieved according to the centrifugal force applied or the similarly acting force. Thus, a liquid can be repeatedly retained and released in said cavity. Furthermore, in the device according to the disclosure, the membrane makes it possible for incubation of a sample or a liquid with a solid phase to be carried out in said cavity. For this purpose, it is possible, for example, for the cavity to contain beads which, as a result of their specific properties, allow, in particular, binding of, for example, a protein to be purified or another molecule.

The German patent application DE 10 2010 003 223 A1 discloses a device having two bodies which are arranged axially on top of one another and have in each case at least one cavity, the bodies being rotatable against one another. Said device provides a position-dependent opening of at least one cavity, use being made in particular of a spike arranged on one body to tear open or pierce a film forming a closure of another cavity and to thus release liquid. In contrast, the solution according to the disclosure has the advantage that the release of the liquid or the opening of a cavity can be carried out repeatedly or is reversible. Depending on the centrifugal force applied or the similarly acting force, liquid can be released from a cavity or the liquid remains in the cavity.

Owing to the valve action of the membrane used according to the disclosure, the device according to the disclosure can be used in a highly variable manner and be adapted and set up for very different protocols to be carried out. In particular, it is possible, for example, to carry out repeated application of sample and passage of different buffers on a bead matrix, the corresponding beads being situated in the cavity having the membrane. By using, according to the disclosure, the membrane as, for example, a closure of the base of reagent storage cavities, it is possible, through application of corresponding centrifugal forces or similarly acting forces, to release a liquid repeatedly, in principle as often as desired, from the cavity and to transfer said liquid along the intended fluid path, for example into a reaction cavity.

Compared to a device in which liquid is released by mechanical destruction of a closure film, the device according to the disclosure has furthermore the advantage that the membrane according to the disclosure exhibits the action of a repeatedly closeable valve, and so, with appropriate control or design of the device and the centrifugation protocol, the released liquid can be retained for a particular duration in a defined space and is then forwarded again along the intended fluid path. It is thus readily possible for multiple liquids to be incubated together. In addition, the incubation of the liquid with a solid phase is possible, the solid phase being formed by, for example, beads which allow, in a manner known per se, very effective purification of a target molecule from a solution. Thus, using the device according to the disclosure, it is possible for bead-based purification protocols in particular to be carried out without additional complexity in terms of apparatus. A major advantage is that it is possible to completely dispense with costly magnetic beads, which would require corresponding specific magnetic devices for removal from the solution.

The valve action of the membrane is achieved by the dependence of membrane permeability on pressure. It is useful to select the membrane such that the pressure of the liquid column at low centrifugal forces or similarly acting forces is below the pressure required for penetration of the membrane by the particular liquid. Consequently, the “valve” is closed at low centrifugal force. When the centrifugal force is elevated, the pressure increases, and so the liquid passes through the membrane. The “valve” is thus open or permeable at elevated centrifugal force. The dependence of permeability on pressure can be established and adapted according to the particular application by using an appropriate membrane. For example, it may be preferable for the membrane to be impermeable to liquids at a gravitational acceleration of <100 g and to be permeable to liquids at a gravitational acceleration of >100 g. Here, g refers to the gravity of Earth. The pore size of the membrane can, for example, be selected such that the pore size is 10 μm or smaller, more particularly 5 μm or smaller, or 1 μm or smaller. When using beads in the cavity having the membrane, it is useful for the pore diameter of the membrane to be smaller than the diameter of the beads.

Besides porosity, pore size and pore geometry, the permeability properties of a membrane are also dependent on the material of the membrane. The materials selected for the membrane may have hydrophilic or hydrophobic properties depending on the particular application. For example, silica membranes or polymer membrane are useful. Owing to their absorption properties, membranes composed of PVDF (polyvinylidene fluoride) are suitable especially for protein applications. In addition, the wetting properties of the membrane may also be important, and so, when selecting an appropriate membrane, the properties of the liquid which is to be retained or flushed out by the membrane may also have some influence.

The membrane in the device according to the disclosure can, for example, be mounted on a porous support structure, for example a glass frit. The membrane can be fixed and/or sealed using mechanical means, for example using a sealing ring.

In the device according to the disclosure, intrusion of guide tongues of one body into a row of profiled teeth of the other body is particularly envisaged for the rotating of the bodies against one another. According to the disclosure, the term “tongue” is understood to mean a tongue in a tongue-and-groove joint. In addition, a restoring means, more particularly a spring, is envisaged, which acts against the centrifugal force or the similarly acting force. The bodies are arranged in the device such that they are shifted radially outward within a surrounding shell body when a centrifugal force is acting. Upon application of pressurized air in the upper region of the device, the bodies within the shell body are shifted downward. Here, one of the bodies is locked by, for example, a fixing means such that it cannot rotate, but is nevertheless moveable in a radial or downward direction. The other body is rotated owing to the intrusion of the guide tongues into a row of profiled teeth of the locked body in the manner of a ballpoint-pen mechanism. This brings the various cavities of the individual bodies in close proximity to one another in a guided manner, and so the fluid flow can be activated accordingly and the process can be managed by this means.

The disclosure additionally encompasses the use of a reaction container having a membrane arranged therein, the permeability of which to liquids is dependent on an acting centrifugal force or a similarly acting force, for carrying out chemical and/or biochemical processes, more particularly for carrying out automated purification of proteins and/or genetic material. The use according to the disclosure is particularly suitable for the use of bead-based purification protocols, the membrane exercising a restraining function for the beads. According to the disclosure, bead-based purification protocols can be carried out with very low complexity in terms of apparatus.

Owing to the above-described valve action of the membrane arranged in the reaction container, the reaction container can be used in a very advantageous manner for a very wide variety of different protocols for purifying biological or other molecules. Particularly preferably, the reaction container is used as part of the above-described device according to the disclosure.

Further features and advantages of the disclosure are revealed by the following description of exemplary embodiments in connection with the drawings. Here, the individual features can in each case be realized separately or in combination with one another.

### BRIEF DESCRIPTION OF THE DRAWINGS

In the drawings:

FIG. 1 shows a diagrammatical sectional view of a cavity having a membrane as a section from a preferred embodiment of a device according to the disclosure and

FIG. 2 shows a diagrammatical sectional view of a preferred design of a device according to the disclosure.

### DETAILED DESCRIPTION

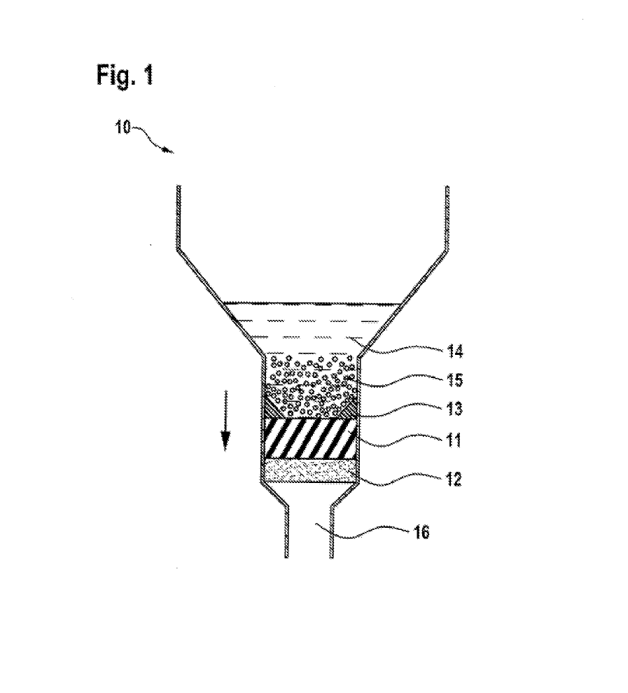

FIG. 1 shows a cavity having a membrane as the element essential to the disclosure of a device according to the disclosure. The cavity is designed as a reaction container 10, a funnel-shaped taper of the reaction container 10 being envisaged in the direction of the fluid flow, which is indicated by an arrow. Arranged in a cylindrical segment within the taper of the reaction container 10 is a membrane 11 which occupies the cross section of the container at this point. Situated below the membrane 11 is a porous support or supporting structure 12, on which the membrane 11 is stably mounted. The porous supporting structure 12 used can, for example, be glas frits. Suitable materials for the membrane 11 are especially materials which exhibit low absorption with respect to the substance to be purified. In the case of protein purification, PVDF membranes can be used for example. A sealing ring 13 is provided at the points of contact of the membrane 11 with the wall of the reaction vessel in order to avoid unwanted fluid flow. A liquid column 14 is depicted above the membrane 11. The liquid contains beads 15. It is useful for the pore diameter of the membrane 11 to be smaller than the dimensions of the substances to be retained, i.e. more particularly the beads 15. The pore diameter is preferably below 10 μm. For example, a pore diameter of <1 μm, for example 0.45 μm, is especially useful. The beads used can, for example, be silica beads, nickel beads, polymer beads or glass beads. Depending on the particular application, for example depending on the molecule to be purified, certain surface properties of the beads are utilized. For example, it is possible to use native beads which, owing to their material-intrinsic binding properties, have an affinity for the particular molecule to be purified. In other cases, additional binding chemistries can be used. For example, certain interaction partners of the target molecules to be isolated can be coupled to the beads in order to thus achieve binding of the target molecule to the beads.

The beads 15 are mounted directly on the membrane 11 (filter membrane). Depending on the acting centrifugal force or similarly acting force, the membrane is impermeable or permeable to liquids. Thus, when an appropriate centrifugal force or similarly acting force is exerted, the liquid 14 penetrates the membrane 11 and the anyway permeable support 12 and is discharged along the fluid guide 16. During this operation, the beads 15 are retained. Here, the direction of the fluid flow, which is indicated by the arrow, also corresponds to the direction of the acting centrifugal force or the similarly acting force.

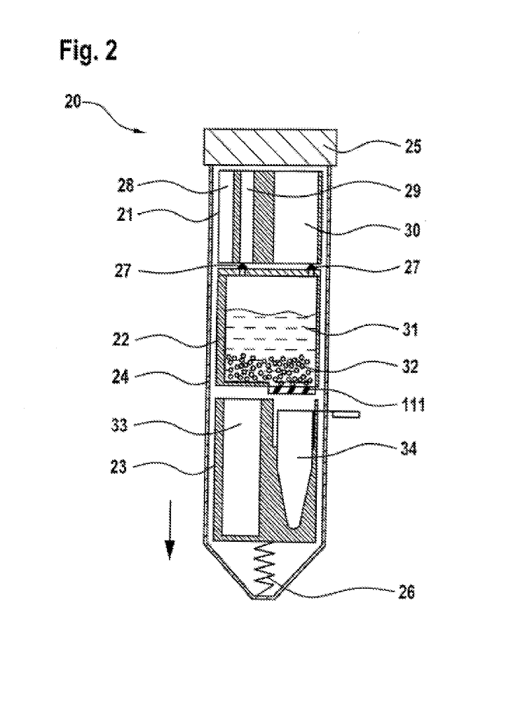

The “membrane valve” according to the disclosure is suitable for various applications, especially in connection with automated protein purification and/or DNA or RNA purification. In particular, it can be used in centrifugal devices, especially in a system in which two or more bodies arranged axially on top of one another and having in each case a cavity are present and in which the fluid flow for automated performance of a purification protocol is realized by rotation of the bodies against one another. FIG. 2 shows such a centrifugal system 20 as an example of a device according to the disclosure. In this embodiment, three bodies 21, 22 and 23 which are rotatable against one another are provided. The bodies 21, 22 and 23 can also be referred to as turrets, since they each have one or more cavities which can be situated in different positions relative to one another. Here, the turret 21 and turret 23 are locked within a surrounding shell body 24 by suitable guide means, and so only the turret 22 can rotate with respect to the turret 21 and the turret 23. From its external shape, the shell body 24 corresponds to a customary centrifugation tube, having for example a volume of 50 ml. The shell body 24 is closable using a lid 25. Within the shell body 24, the turrets 21 and 22 and, optionally, also the turret 23 are arranged in such a way that they are shiftable in a radial direction, indicated by an arrow, as a result of an acting centrifugal force. This means that, in the event of an acting centrifugal force, the bodies 21, 22 and 23 are shifted in the direction of the arrow, i.e. downward in this representation. This shift acts against the restoring force of a restoring means 26, which is provided in this representation as a spring 26 in the lower, conical end of the shell body 24. In the event of diminishing centrifugal force, the spring 26 brings about movement of the turrets 21, 22 and 23 back to their starting position. Suitable guide means (not depicted) especially on the turrets 21 and 22 bring about rotating of the second turret 22 with respect to the turrets 21 and 23. For this purpose, an integrated “ballpoint-pen mechanism” in particular can be used. Mechanical means on the turret 21, more particularly spikes 27, bring about the tearing open of cavities 28, 29 or 30 in the turret 21, depending on the position of the turret 22 in relation to the turret 21. This is realized by the cavities 28, 28 and 30 being provided with a pierceable film in their (in this representation) lower region. As a result of tearing by the spikes 27, the liquid contained in each of cavities 28, 29 and 30 is released in the direction of the arrow. Here, it is possible, for example, for different reagents to be contained in the cavities 28 and 29. The cavity 30 can, for example, store a cell lysate as sample, in which the molecule to be purified, for example a protein, is situated. The second turret 22 has a central cavity as incubation chamber 31. The sample, i.e. the lysate, and also, according to the purification protocol, various reagents are introduced into said reaction chamber. In addition, the beads 32 are situated in the incubation chamber 31. The interaction between the beads 32 as solid phase and the molecule to be purified from the liquid sample takes place here. In the direction of the fluid flow, the reaction chamber 31 is delimited in one region by a membrane 111. The membrane 111 can, for example, have a diameter of from 1 to 7 mm, more particularly in line with the dimension of the device according to the disclosure. According to the disclosure, said membrane 111 is permeable or impermeable to liquids on the basis of the acting centrigual force or similarly acting force, and so the membrane 111 can be utilized as a “valve” in order to be able to open the incubation chamber 31 for passage of the liquid. The membrane 111 is only provided in a particular demarcated region of the base of the incubation chamber 31. The rest of the region is impassable to liquids. Depending on the orientation of the turret 22, the permeable region having the membrane 111 is situated in close proximity to or above a particular cavity of the third turret 23. In this embodiment, two cavities are present in the turret 23. The cavity 33 is intended for waste liquid. The cavity 34 is intended for accommodating a customary reaction vessel, for example an Eppendorf reaction vessel, in which an eluate from the incubation chamber 31 can be collected, the eluate containing the purified molecule. The possibility of switching the reaction vessel in the cavity 34 makes it possible here to collect various fractions from the purification process, which fractions can be appropriately analyzed and/or further processed.

The device 20 according to the disclosure is, for example, suitable for carrying out bead-based protein purification. The beads 32 are situated in the incubation chamber 31 and various solutions and reagents from the turret 21 are applied thereto. The sequence is controlled by rotating of the turret 22 in relation to the turret 21 and to the turret 23, the reagents and other solutions being released from the turret 21 in the desired manner into the incubation chamber 31 owing to the spikes 27. By means of the orientation of the turret 22 in relation to the turret 23, the liquid is transferred from the incubation chamber 31 into the cavities or vessels in the turret 23. This fluid flow is controlled by the membrane 111, which acts as a valve. The “valve” is controlled by the centrifugal force applied. Here, the general sequence of the protocol comprises the beads firstly being washed with an aqueous solution in order to set the appropriate pH at the bead surface. Subsequently, the cell lysate, which contains the protein to be purified, is added and incubated with the beads, and so specific binding of the proteins to the beads occurs. After the incubation, unbound lysate is flushed out across the membrane 111 using wash buffer before specific elution is carried out using an elution buffer. In this manner, it is possible, for example, to purify a His-tag protein, i.e. a recombinant protein provided with a histidine tag, from a corresponding bacterial lysate. For this purpose, it is possible to use nonmagnetic beads which have a diameter of, for example, >5 μm and are provided with nickel, e.g. Ni-NTA beads. The beads are transferred from one of the storage chambers 28 or 29 into the incubation chamber 31. The purification is processed analogously to a manual protocol, the required liquids being stored in the cavities of the turret 21 and released when required and being transferred into the incubation chamber 31. The rinsing or washing and elution of the beads is carried out by means of the valve action of the membrane 111, the membrane retaining the delivered liquid at gravitational accelerations<100 g and allowing the liquid to completely pass through the membrane at gravitational accelerations>100 g and no residual volume remaining in the membrane pores. The liquid can be incubated in the idle state of the centrifuge or at low gravitational accelerations, more particularly at <100 g.

An appropriate procedure is shown in the table below.

| | | | Centrifu- |
|---|---|---|---|
| Step | Action | Time | gation |
| Loading with | Addition of 100 μl of | | |
| the beads | bead suspension | | |
| | Rinsing out | 60 s | 1500 g |
| Buffering | 250 μl of wash buffer | 5 s | 10 g |
| | Incubation | 55 s | 40 g |
| | Rinsing out | 60 s | 1500 g |
| Addition of | 500 μl of lysate | 5 s | 10 g |
| the lysate | Incubation | 55 s/29 min | 40 g/10 g |
| | Rinsing out | 60 s | 6000 g |
| Wash I | 250 μl of wash buffer | 5 s | 10 g |
| | Incubation | 55 s | 40 g |
| | Rinsing out | 60 s | 6000 g |
| Wash II | 250 μl of wash buffer | 5 s | 10 g |
| | Incubation | 55 s | 40 g |
| | Rinsing out | 60 s | 6000 g |
| Wash III | 250 μl of wash buffer | 5 s | 10 g |
| | Incubation | 55 s | 40 g |
| | Rinsing out | 60 s | 6000 g |
| Elution | 100 μl of elution buffer | 5 s | 10 g |
| | Incubation | 55 s/60 s | 40 g/10 g |
| | Rinsing out | 60 s | 6000 g |

The starting point for the development of an appropriate procedure can be a manual protocol, the individual protocol steps being adapted to the beads used and the membrane used.

When carrying out the described purification protocol, it should be noted that the permeability of the membrane may alter over the course of the protocol, since the pore system of the membrane may be supplemented, through the addition of the lysate, by particles and agglomerates from the lysate. This may result in strengthening of the restraining action of the membrane.

The device according to the disclosure is in principle suitable for all types of bead-based purification, for example it is possible to purify various proteins, peptides, DNA or RNA from a sample. For this purpose, it is merely necessary to select beads which are appropriate in each case and to appropriately pretreat the beads with corresponding interaction partners of the substance to be purified in each case and/or to adapt the binding chemistries in a manner known per se.

In other designs of the devices according to the disclosure, further membranes having a valve function can be used in the device so that the fluid flow can also be effected between other cavities, for example between reagent storage cavities and an incubation chamber.

What is claimed is:

1. A device for carrying out chemical and/or biochemical processes, comprising:
at least two bodies arranged axially on top of one another and each defining at least one cavity,
wherein the at least two bodies are rotatable against one another on the basis of a centrifugal force or a similarly acting force,
wherein one body of the at least two bodies includes a first cavity of the at least one cavity which includes a membrane, the permeability of which to liquids is dependent on an acting centrifugal force or a similarly acting force.

2. The device according to claim 1, further comprising:
beads arranged in the first cavity having the membrane.

3. The device according to claim 1, wherein the membrane is impermeable to liquids at a gravitational acceleration of less than 100 g and permeable to liquids at a gravitational acceleration of greater than 100 g.

4. The device according to claim 1, wherein a pore size of the membrane is 10 μm or smaller.

5. The device according to claim 1, wherein the membrane includes a polyvinylidene fluoride membrane.

6. The device according to claim 1, further comprising:
a porous support structure on which the membrane is mounted.

7. The device according to claim 1, wherein:
a first body of the at least two bodies includes at least one guide tongue,
a second body of the at least two bodies includes a row of profiled teeth,
the at least one guide tongue intrudes into the row of profiled teeth, and
a restoring force on the at least two bodies acts against the centrifugal force or against the similarly acting force.

8. A method of using of a reaction container the method comprising:
carrying out at least one of a chemical process and a biochemical process using a reaction container including a membrane arranged therein having a permeability to liquids that is dependent on an acting centrifugal force or a similarly acting force.

9. The method according to claim 8, wherein the at least one of the chemical process and the biochemical process is bead-based.

10. The method according to claim 8, wherein:
the reaction container includes at least two bodies arranged axially on top of one another, each defining at least one cavity,
the at least two bodies are rotatable against one another on the basis of the centrifugal force or the similarly acting force, and
a first cavity of the at least one cavity includes the membrane.

11. The device according to claim 4, wherein the pore size of the membrane is 5 μm or smaller, preferably 1 μm or smaller

12. The device according to claim 11, wherein the pore size of the membrane is 1 μm or smaller

13. The method of claim 8, the carrying out of the at least one of the chemical process and the biochemical process including carrying out automated purification of at least one of proteins and genetic material using the reaction container including the membrane arranged therein having a permeability to liquids that is dependent on an acting centrifugal force or a similarly acting force.
